(19) United States
(12) Patent Application Publication
(10) Pub. No.: US 2026/0041766 A1
(43) Pub. Date: Feb. 12, 2026

(54) SYSTEMS AND METHODS FOR PRODUCING ANTIBODIES

(71) Applicant: LUBERSKI, INC. DBA HIDDEN VILLA RANCH, Fullerton, CA (US)

(72) Inventor: Timothy LUBERSKI, Yorba Linda, CA (US)

(73) Assignee: LUBERSKI, INC. DBA HIDDEN VILLA RANCH, Fullerton, CA (US)

(21) Appl. No.: 19/100,245

(22) PCT Filed: Aug. 2, 2023
(86) PCT No.: PCT/US2023/029336

§ 371 Date: Jan. 31, 2025

Related U.S. Application Data
(60) Provisional application No. 63/394,967, filed on Aug. 3, 2022.

(51) Int. Cl. A61K 39/42; A61K 35/57; A61K 39/00; A61K 39/215; C07K 16/02; C07K 16/10; G01N 33/53

(52) U.S. Cl. CPC A61K 39/42; A61K 35/57; A61K 39/215; C07K 16/02; C07K 16/1003; G01N 33/53; A61K 2039/542; A61K 2039/545; A61K 2039/552; C07K 2317/11; C07K 2317/23

(57) ABSTRACT

Systems, methods for producing antibodies are disclosed. A method for increasing IgY in a bird egg includes exposing a bird to one or more antigens where the exposing is performed on at least two separate occurrences and where a time between the at least two separate occurrences is at least one week.

### CROSS REFERENCE TO PRIOR APPLICATION

This application is a United States national stage application for PCT/US2023/029336, entitled as SYSTEMS AND METHODS FOR PRODUCING ANTIBODIES, filed Aug. 2, 2023, which claims the benefit of U.S. Provisional Ser. No. 63/394,967, entitled as “SYSTEMS AND METHODS FOR PRODUCING ANTIBODIES”, filed Aug. 3, 2022, which is incorporated by reference in its entirety.

### FIELD OF THE INVENTION

This disclosure relates to the fields of antibody production and chicken egg production.

### BACKGROUND

When chicks are immunized, they produce antibodies that are detectable in their blood and in eggs that the chicks lay later on. Chickens that are immunized with an antigen will typically continue to lay eggs that contain the antibodies for the rest of their lives. Thus, a single immunization may yield a multitude of antibody containing eggs. The antibodies, or immunoglobulin, is abbreviated IgY in birds and IgG in mammals.

The IgY antibodies will target the antigen used to immunize the chick. IgY antibodies may be extracted from eggs that are laid by immunized chickens and used to inoculate mammals. IgY antibodies from chicken eggs are inexpensive to produce and are considered preferable in many aspects to other forms of immunization such as antibiotics.

But even though IgY antibodies, which are extracted from the eggs of immunized chickens, may be inexpensive to produce, plentiful, and relatively easy to administer, they are relatively underutilized for remedying various diseases caused by pathogens. There is a continual need for inexpensive remedies for various pathogens that make use of IgY antibodies.

### SUMMARY

Methods and systems for producing antibodies are disclosed. An exemplary embodiment is a method for increasing IgY in a bird egg. The method includes exposing a bird to one or more antigens where the exposing is performed on at least two separate occurrences A time between the at least two separate occurrences is at least one week. The method may further include determining an amount of IgY in blood of the bird prior to exposing the one or more antigens on at least one occasion where a time of the exposing the one or more antigens on at least one occasion is based on the amount of IgY in the blood. Determining an amount of IgY may be performed at least twice. Determining an amount of IgY may be performed at a frequency of between about once every day and once every 4 weeks. A frequency between the at least two separate occurrences of the exposing the one or more antigens may be between about one time a month and about one time every two months. The one or more antigens may include infectious bronchitis virus (IBV). The method may further include administering the bird egg to a human patient or animal subject. The administering may include oral administration of the bird egg. The bird egg may have about 20% more IgY antibodies than control eggs. The IgY antibodies may be used to inoculate against avian infectious bronchitis virus (IBV) in the human patient or animal subject. The IgY antibodies may be used to inoculate against disease within a gastrointestinal tract in the human patient or animal subject. The disease within the gastrointestinal tract may be selected from the group consisting of tooth decay, periodontitis and gingivitis, gastritis and gastric ulcer, celiac disease, oral thrush, and infant rotavirus diarrhea.

Another general aspect is an IgY delivery system. The system includes an egg that is laid by a bird that were exposed to one or more contagions. The egg is from a bird that was exposed to the one or more contagions on two or more occasions. At least one of the two or more occasions may occur more than about 16 weeks after the bird hatched. An amount of IgY in blood of the bird may be tested prior to at least one of the two or more occasions. A time of at least one of the two or more occasions may be based on the amount of IgY in the blood of the bird. The time of at least one of the two or more occasions may be triggered when an IgY concentration in the blood dropped below about 13 mg/mL. The egg may be prepared by purifying IgY from yolk of the egg where the IgY is bound to at least one of S1 and S2 subunits from a Delta variant of the COVID virus. The one or more contagions may include avian infectious bronchitis virus (IBV). The bird may be verified to contain IgY antibodies in its blood prior to laying the egg.

An exemplary embodiment is a method of producing IgY. The method includes administering a contagion to a bird, the contagion comprising avian infectious bronchitis virus (IBV) two or more times and feeding the bird a high-nutrient diet comprising vitamin A, vitamin D, and vitamin E. The method further includes collecting eggs from the bird and extracting the IgY from the eggs. A concentration of vitamin A may be between about 10,000 IU per kg of the high-nutrient diet and about 200,000 UI per kg of the high-nutrient diet. A concentration of vitamin D may be between about 500 IU per kg of the high-nutrient diet and about 10,000 UI per kg of the high-nutrient diet. A concentration of vitamin E may be between about 1,000 mg per kg of the high-nutrient diet and about 10,000 mg per kg of the high-nutrient diet.

### BRIEF DESCRIPTION OF THE DRAWINGS

FIG. 1 illustrates an embodiment of a process for immunizing an egg laying chicken with a contagion to produce IgY containing eggs.

FIG. 2A is an ELISA assay showing reactivity of the receptor binding domain (RBD) for SARS-CoV-2 and IgY antibodies derived from various sources including unimmunized purified eggs via size exclusion chromatography (SEC), immunized purified eggs via SEC, un-immunized crude eggs, and immunized crude eggs.

FIG. 2B is an ELISA assay showing reactivity of the soluble spike trimer for SARS-CoV-2 and IgY antibodies derived from various sources including unimmunized purified eggs via size exclusion chromatography (SEC), immunized purified eggs via SEC, un-immunized crude eggs, and immunized crude eggs.

FIG. 3A is a schematic showing a system for producing IgY boosted eggs via a hyperimmunization program.

FIG. 3B is an image of an SDS-PAGE analysis in reducing agent comparing various commercial IgY samples with ovalbumin collected from hens that were immunized according to the disclosed subject matter.

FIG. 3C is an image of an SDS-PAGE analysis in non-reducing agent comparing various commercial IgY samples with ovalbumin collected from hens that were immunized according to the disclosed subject matter.

FIG. 3D is an ELISA assay showing reactivity of the receptor binding domain (RBD) for SARS-CoV-2 and IgY antibodies derived from various sources including un-immunized purified eggs via size exclusion chromatography (SEC), immunized purified eggs via SEC, un-immunized crude eggs, and immunized crude eggs.

FIG. 3E is an embodiment of a process for creating a pseudo-virus neutralization assay, with which to test effectiveness of derived IgY, from immunized hens, for neutralizing SARS-CoV-2.

FIG. 4A is an ELISA assay showing reactivity of the receptor binding domain (RBD) for SARS-CoV-2 and immunoglobulin samples representing human IgG1, IgY derived from grocery store eggs (control), eggs from hens that were supplemented with vitamin D and immunized to avian influenza, eggs from hens that were supplemented with vitamins B1, B2, and B3 and immunized to the recombinant receptor binding domain (rRBD) of SARS-CoV-2 (B1-B2-B3 rRBD immunized), and pasteurized eggs that were immunized to avian influenza.

FIG. 4B is an ELISA assay showing reactivity of the soluble spike trimer for SARS-CoV-2 and immunoglobulin samples representing human IgG1, IgY derived from grocery store eggs (control), eggs from hens that were supplemented with vitamin D and immunized to avian influenza, eggs from hens that were supplemented with vitamins B1, B2, and B3 and immunized to the recombinant receptor binding domain (rRBD) of SARS-CoV-2 (B1-B2-B3 rRBD immunized), and pasteurized eggs that were immunized to avian influenza.

FIG. 5A is an SDS-PAGE analysis of immunoglobulin samples taken from eggs laid by immunized hens.

FIG. 5B is a table showing a yield of IgY from eggs laid by hens from a control group.

FIG. 5C is a table showing a yield of IgY from eggs laid by hens that were immunized and fed a diet based on an embodiment of the disclosed subject matter.

### DETAILED DESCRIPTION

The disclosed subject matter is a recently developed IgY-boosted shell egg from chickens that is specially formulated to strengthen the immune system of an individual. Besides chickens, the disclosed subject matter may be practiced with other forms of poultry or agricultural birds including but not limited to turkeys, ducks, geese, pheasants, guinea fowls, quails, squabs, and ostriches. Embodiments herein with references to any type of agricultural bird are not intended be limited to a single species, but instead may be applicable to many or all agricultural birds.

In recent controlled tests, the boosted eggs in chickens were demonstrated to contain 20% more IgY immunoglobulin antibodies than commercial eggs tested. Additionally, they contain high bioavailable vitamin D to restore seasonal deficiencies. The IgY-boost hens are fed a high-nutrient dietary supplement and undergo a boosted vaccination regime which results in the eggs that contain increased levels of immuno-proteins which strengthen the immune system and decrease the chance of viral infection and transmission of viruses.

The IgY immunoglobulin antibodies are present in the yolk of these specially produced IgY-boost eggs. Much like how colostrum in mother's milk provides passive immunity and helps to develop a stronger immune system, the immunoglobins in the yolk of a hen's egg provides the same benefits to its chicks. Oral ingestion of those same immunoglobins and micronutrients in eggs can provide significant health benefits.

Chicken antibodies, known as IgY immunoglobulins are similar in structure to IgG antibodies and compatible for human use, but more stable than IgG antibodies and exhibit survival during passage through the GI track. Egg immunoglobulin is a food component and has received Generally Recognized as Safe (GRAS) status from both the FDA and USDA. Egg antibodies have been used to provide passive immunity against a wide range of bacteria and viruses for many decades and unlike other non-human mammalian antibodies, IgYs do not trigger an inflammatory response.

Egg-derived antibodies are polyclonal and differ from monoclonal antibodies in that they contain multiple antibody species which can cross react with multiple epitopes from related species; thus, veterinary immunogens can provide protection against human pathogens. Ingestion of the eggs containing IgY antibodies have been shown to be effective in prevention and treatment of disease in various locations within the gastrointestinal tract including tooth decay, periodontitis and gingivitis, gastritis and gastric ulcer, celiac disease, oral thrush, and infant rotavirus diarrhea. IgY therapy has also been successfully used to treat systemic infection in humans and animals. Egg yolk immunoglobulin has also been used in oral applications to reduce the incidence and severity of respiratory disease in animal models and in clinical trials.

Recent results from a controlled study indicate that the IgY-boost eggs contain an average of 20% more IgY antibody than commercial eggs. When hens are immunized against a pathogen, the IgY antibodies are produced in the blood serum and passed to the embryo through the egg yolk. The IgY-eggs are collected from hens fed specially formulated high nutrient diets containing selenium, vitamins A, & D. The IgY hens are treated with a specialized boosted immunization schedules against a range of veterinary viruses, including avian infectious bronchitis virus (IBV), a chicken coronavirus. In fact, the Spike protein S1 and S2 domains share 58% amino acid identity (excluding the receptor binding domain). Recent studies demonstrate that the anti-IBV antibodies in IgY-eggs strongly cross react and with SARS CoV2 spike protein. Moreover, the data show potent binding of the IgY.

### Sars Cov2 Application

Avian Infectious Bronchitis Virus is a single stranded RNA gamma-coronavirus closely related to the SARS CoV2 beta-coronavirus, with their Spike proteins containing 58% identical amino acids. Conservation of epitopes across contagion species renders polyclonal antibodies effective against multiple pathogens. Recent studies conducted indicate that the polyclonal IgY antibodies present in the eggs not only effectively bind the SARS CoV2 Spike protein, but also can neutralize the virus at nanomolar inhibitor concentrations (Kᵢ). Experimental studies indicate that 67% of SAR CoV2 virus is inactivated in vitro at 1/15 the IgY concentration found in the egg yolk (1 mg/ml).

Egg-laying hens produce high concentrations of polyclonal IgY antibodies which can be readily recovered from egg yolks. The polyclonal hen IgY antibodies are multivalent and will bind to multiple epitopes (antibody binding sites) on multiple antigens from multiple pathogens. IgY antibodies are similar in structure to human IgG antibodies and compatible with human use. In contrast to other non-human mammalian antibodies, avian IgYs do not bind Protein A, Protein G or interact with cellular Fc receptors; furthermore, IgY does not activate the complement system and has been demonstrated to not mediate inflammatory responses in humans.

In various embodiments, chickens are immunized two or more times with a same contagion. In an exemplary embodiment, chickens are immunized at a frequency of about once every week. In an exemplary embodiment, chickens are immunized at a frequency of about once every two weeks. In an exemplary embodiment, egg laying chickens are immunized at a frequency of about once every month. In an exemplary embodiment, chickens are immunized at a frequency of about once every two months. In an exemplary embodiment, chickens are immunized at a frequency of about once every three months. In an exemplary embodiment, chickens are immunized at a frequency of about once every four months. In an exemplary embodiment, chickens are immunized at a frequency of about once every six months. In an exemplary embodiment, chickens are immunized at a frequency of about once every eight months. In an exemplary embodiment, chickens are immunized at a frequency of about once every nine months. In an exemplary embodiment, chickens are immunized at a frequency of about once every twelve months.

In various embodiments, at least one of the immunizations occurs between about 16 weeks after the chicken hatches and about 20 weeks after the chicken hatches. In various embodiments, at least one of the immunizations occurs between about 12 weeks after the chicken hatches and about 20 weeks after the chicken hatches. In various embodiments, at least one of the immunizations occurs about 14 weeks after the time that the chicken hatches. In various embodiments, at least one of the immunizations occurs about 16 weeks after the time that the chicken hatches. In various embodiments, at least one of the immunizations occurs about 18 weeks after the time that the chicken hatches. In various embodiments, at least one of the immunizations occurs about 20 weeks after the time that the chicken hatches.

In an exemplary embodiment, immunized egg laying hens are tested for antibodies on a regular basis such as once a month. The hens are further immunized based on the antibody tests. For instance, the hens are further immunized if their antibody tests indicate that antibody levels drop below a threshold. In one example antibody levels are measured as an antibody concentration. In another example, antibody levels are measured as a total yield of IgY antibodies.

For instance, immunized egg laying hens may be further immunized if an average total IgY in eggs drops below about 100 mg. In various embodiments, immunized egg laying hens are further immunized if an average total IgY in eggs drops below about 110 mg. In various embodiments, immunized egg laying hens are further immunized if an average total IgY in eggs drops below about 120 mg. In various embodiments, immunized egg laying hens are further immunized if an average total IgY in eggs drops below about 130 mg. In various embodiments, immunized egg laying hens are further immunized if an average total IgY in eggs drops below about 140 mg. In various embodiments, immunized egg laying hens are further immunized if an average total IgY in eggs drops below about 150 mg. In various embodiments, immunized egg laying hens are further immunized if an average total IgY in eggs drops below about 160 mg.

### Diet to Increase Igy in Eggs

In various embodiments, immunized chickens are fed a diet that is optimized to increase IgY antibodies in eggs produced by the chickens. In various embodiments, chickens are fed a diet that is supplemented with at least one of selenium, vitamin A, vitamin D, and vitamin E. In an exemplary embodiment, chickens are fed a diet that is supplemented with at least two of selenium, vitamin A, vitamin D, and vitamin E. In an exemplary embodiment, chickens are fed a diet that is supplemented with at least three of selenium, vitamin A, vitamin D, and vitamin E. In an exemplary embodiment, chickens are fed a diet that is supplemented with at least two of selenium, vitamin A, vitamin D, and vitamin E.

In various embodiments, a vitamin D concentration in any of the diets disclosed herein may be between about 50 IU VD₃/kg of diet and 250 IU VD₃/kg of diet. The denominator in the unit IU VD₃/kg represents a mass of feed that is administered to a chicken. In various embodiments, a vitamin D concentration in any of the diets disclosed herein may be between about 250 IU VD₃/kg and 500 IU VD₃/kg. In various embodiments, a vitamin D concentration in any of the diets disclosed herein may be between about 500 IU VD₃/kg and 750 IU VD₃/kg. In various embodiments, a vitamin D concentration in any of the diets disclosed herein may be between about 750 IU VD₃/kg and 1000 IU VD₃/kg. In various embodiments, a vitamin D concentration in any of the diets disclosed herein may be between about 1000 IU VD₃/kg and 1250 IU VD₃/kg. In various embodiments, a vitamin D concentration in any of the diets disclosed herein may be between about 1250 IU VD₃/kg and 1500 IU VD₃/kg. In various embodiments, a vitamin D concentration in any of the diets disclosed herein may be between about 1500 IU VD₃/kg and 1750 IU VD₃/kg. In various embodiments, a vitamin D concentration in any of the diets disclosed herein may be between about 1750 IU VD₃/kg and 2000 IU VD₃/kg. In various embodiments, a vitamin D concentration in any of the diets disclosed herein may be between about 2000 IU VD₃/kg and 2250 IU VD₃/kg. In various embodiments, a vitamin D concentration in any of the diets disclosed herein may be between about 2250 IU VD3/kg and 2500 IU VD₃/kg. In various embodiments, a vitamin D concentration in any of the diets disclosed herein may be between about 2500 IU VD₃/kg and 2750 IU VD₃/kg. In various embodiments, a vitamin D concentration in any of the diets disclosed herein may be between about 2750 IU VD₃/kg and 3000 IU VD₃/kg.

In various embodiments, a selenium concentration in any of the diets disclosed herein may be between about 0.05 mg Se/kg diet and 0.10 mg Se/kg diet. In various embodiments, a selenium concentration in any of the diets disclosed herein may be between about 0.10 mg Se/kg and 0.15 mg Se/kg. In various embodiments, a selenium concentration in any of the diets disclosed herein may be between about 0.15 mg Se/kg and 0.20 mg Se/kg. In various embodiments, a selenium concentration in any of the diets disclosed herein may be between about 0.20 mg Se/kg and 0.25 mg Se/kg. In various embodiments, a selenium concentration in any of the diets disclosed herein may be between about 0.25 mg Se/kg and 0.30 mg Se/kg. In various embodiments, a selenium concentration in any of the diets disclosed herein may be between about 0.30 mg Se/kg and 0.40 mg Se/kg. In various embodiments, a selenium concentration in any of the diets disclosed herein may be between about 0.40 mg Se/kg and 0.50 mg Se/kg. In various embodiments, a selenium concentration in any of the diets disclosed herein may be between about 0.50 mg Se/kg and 0.60 mg Se/kg. In various embodiments, a selenium concentration in any of the diets disclosed herein may be between about 0.60 mg Se/kg and 0.70 mg Se/kg. In various embodiments, a selenium concentration in any of the diets disclosed herein may be between about 0.70 mg Se/kg and 0.80 mg Se/kg. In various embodiments, a selenium concentration in any of the diets disclosed herein may be between about 0.80 mg Se/kg and 0.90 mg Se/kg. In various embodiments, a selenium concentration in any of the diets disclosed herein may be between about 0.90 mg Se/kg and 1.00 mg Se/kg. In various embodiments, a selenium concentration in any of the diets disclosed herein may be between about 1.00 mg Se/kg and 1.20 mg Se/kg. In various embodiments, a selenium concentration in any of the diets disclosed herein may be between about 1.20 mg Se/kg and 1.40 mg Se/kg. In various embodiments, a selenium concentration in any of the diets disclosed herein may be between about 1.40 mg Se/kg and 1.60 mg Se/kg.

In various embodiments, a vitamin A concentration in any of the diets disclosed herein may be between about 10,000 IU/kg diet and 12,500 UI/kg diet. In various embodiments, a vitamin A concentration in any of the diets disclosed herein may be between about 12,500 IU/kg and 15,000 UI/kg. In various embodiments, a vitamin A concentration in any of the diets disclosed herein may be between about 15,000 IU/kg and 17,500 UI/kg. In various embodiments, a vitamin A concentration in any of the diets disclosed herein may be between about 17,500 IU/kg and 20,000 UI/kg. In various embodiments, a vitamin A concentration in any of the diets disclosed herein may be between about 25,000 IU/kg and 30,000 UI/kg. In various embodiments, a vitamin A concentration in any of the diets disclosed herein may be between about 30,000 IU/kg and 40,000 UI/kg. In various embodiments, a vitamin A concentration in any of the diets disclosed herein may be between about 40,000 IU/kg and 50,000 UI/kg. In various embodiments, a vitamin A concentration in any of the diets disclosed herein may be between about 50,000 IU/kg and 60,000 UI/kg. In various embodiments, a vitamin A concentration in any of the diets disclosed herein may be between about 60,000 IU/kg and 70,000 UI/kg. In various embodiments, a vitamin A concentration in any of the diets disclosed herein may be between about 70,000 IU/kg and 80,000 UI/kg. In various embodiments, a vitamin A concentration in any of the diets disclosed herein may be between about 80,000 IU/kg and 90,000 UI/kg. In various embodiments, a vitamin A concentration in any of the diets disclosed herein may be between about 90,000 IU/kg and 100,000 UI/kg. In various embodiments, a vitamin A concentration in any of the diets disclosed herein may be between about 100,000 IU/kg and 120,000 UI/kg. In various embodiments, a vitamin A concentration in any of the diets disclosed herein may be between about 120,000 IU/kg and 140,000 UI/kg. In various embodiments, a vitamin A concentration in any of the diets disclosed herein may be between about 140,000 IU/kg and 160,000 UI/kg. In various embodiments, a vitamin A concentration in any of the diets disclosed herein may be between about 160,000 IU/kg and 180,000 UI/kg. In various embodiments, a vitamin A concentration in any of the diets disclosed herein may be between about 180,000 IU/kg and 200,000 UI/kg. In various embodiments, a vitamin A concentration in any of the diets disclosed herein may be between about 200,000 IU/kg and 250,000 UI/kg. In various embodiments, a vitamin A concentration in any of the diets disclosed herein may be between about 250,000 IU/kg and 300,000 UI/kg. In various embodiments, a vitamin A concentration in any of the diets disclosed herein may be between about 300,000 IU/kg and 350,000 UI/kg. In various embodiments, a vitamin A concentration in any of the diets disclosed herein may be between about 350,000 IU/kg and 400,000 UI/kg.

In various embodiments, a vitamin E concentration in any of the diets disclosed herein may be between about 100 mg/kg of diet and 150 mg/kg of diet. In various embodiments, a vitamin E concentration in any of the diets disclosed herein may be between about 150 mg/kg and 200 mg/kg. In various embodiments, a vitamin E concentration in any of the diets disclosed herein may be between about 200 mg/kg and 250 mg/kg. In various embodiments, a vitamin E concentration in any of the diets disclosed herein may be between about 250 mg/kg and 300 mg/kg. In various embodiments, a vitamin E concentration in any of the diets disclosed herein may be between about 300 mg/kg and 400 mg/kg. In various embodiments, a vitamin E concentration in any of the diets disclosed herein may be between about 400 mg/kg and 500 mg/kg. In various embodiments, a vitamin E concentration in any of the diets disclosed herein may be between about 500 mg/kg and 600 mg/kg. In various embodiments, a vitamin E concentration in any of the diets disclosed herein may be between about 600 mg/kg and 700 mg/kg. In various embodiments, a vitamin E concentration in any of the diets disclosed herein may be between about 700 mg/kg and 800 mg/kg. In various embodiments, a vitamin E concentration in any of the diets disclosed herein may be between about 800 mg/kg and 900 mg/kg. In various embodiments, a vitamin E concentration in any of the diets disclosed herein may be between about 900 mg/kg and 1000 mg/kg. In various embodiments, a vitamin E concentration in any of the diets disclosed herein may be between about 1000 mg/kg and 1500 mg/kg. In various embodiments, a vitamin E concentration in any of the diets disclosed herein may be between about 1500 mg/kg and 2000 mg/kg. In various embodiments, a vitamin E concentration in any of the diets disclosed herein may be between about 2000 mg/kg and 2500 mg/kg. In various embodiments, a vitamin E concentration in any of the diets disclosed herein may be between about 2500 mg/kg and 3000 mg/kg. In various embodiments, a vitamin E concentration in any of the diets disclosed herein may be between about 3000 mg/kg and 4000 mg/kg. In various embodiments, a vitamin E concentration in any of the diets disclosed herein may be between about 4000 mg/kg and 5000 mg/kg. In various embodiments, a vitamin E concentration in any of the diets disclosed herein may be between about 5000 mg/kg and 6000 mg/kg. In various embodiments, a vitamin E concentration in any of the diets disclosed herein may be between about 6000 mg/kg and 7000 mg/kg. In various embodiments, a vitamin E concentration in any of the diets disclosed herein may be between about 7000 mg/kg and 8000 mg/kg. In various embodiments, a vitamin E concentration in any of the diets disclosed herein may be between about 8000 mg/kg and 9000 mg/kg. In various embodiments, a vitamin E concentration in any of the diets disclosed herein may be between about 9000 mg/kg and 10000 mg/kg. In various embodiments, a vitamin E concentration in any of the diets disclosed herein may be between about 10000 mg/kg and 12000 mg/kg. In various embodiments, a vitamin E concentration in any of the diets disclosed herein may be between about 12000 mg/kg and 14000 mg/kg. In various embodiments, a vitamin E concentration in any of the diets disclosed herein may be between about 14000 mg/kg and 16000 mg/kg. In various embodiments, a vitamin E concentration in any of the diets disclosed herein may be between about 16000 mg/kg and 18000 mg/kg. In various embodiments, a vitamin E concentration in any of the diets disclosed herein may be between about 18000 mg/kg and 20000 mg/kg.

### DESCRIPTION OF FIGURES

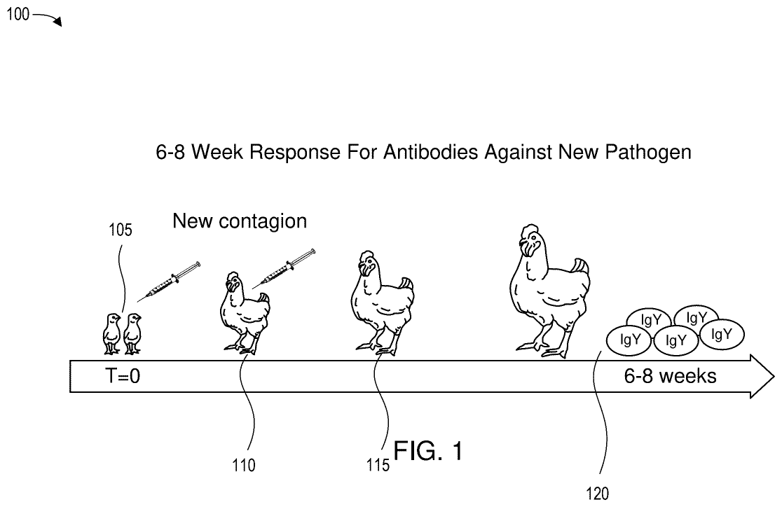

Referring to FIG. 1, FIG. 1 illustrates an embodiment of a process 100 for immunizing an egg laying chicken with a contagion to produce IgY containing eggs. The process 100 is a treatment of egg laying hens that results in eggs that contain more IgY than eggs that are currently produced in the industry. IgY that is collected from the eggs may be administered to human subjects or animals to deliver the IgY antibodies to them. Accordingly, the IgY may be produced efficiently and inexpensively through a modification of established agricultural practices.

Further, administration of the IgY antibodies to a human or animal subject may be performed orally, which decreases the cost of administration. In embodiments that are administered orally, a medical professional is usually not needed to administrate the collected IgY antibodies to the subject. In various embodiments, food may be supplemented with the collected antibodies. Thus, the disclosed subject matter may be practiced to supplement various food products with collected IgY antibodies to confer antibodies to a subject that consumes the food products.

In various embodiments, the collected IgY antibodies are administered by means other than oral administration. For example, the collected IgY antibodies may be administered intranasally whereby the human or animal subject inhales the IgY antibodies through their nasal cavity. In an exemplary embodiment, collected antibodies are configured to be deposited in the nasal cavity of the human or animal subject to confer a passive immunity. The deposited antibodies may then bind to pathogens that enter the nasal cavity. Accordingly, airborne pathogens may be neutralized when they enter the nasal cavity, thus preventing them from dispersing, replicating, or otherwise causing damage in the body.

In various embodiments, a combination of passive immunity and other forms of treatment may be performed concurrently on a human or animal subject. For example, intranasal immunization may be administered to individuals that test positive for a disease. The passive immunity may slow progression of some diseases and/or reduce the transmissibility of the disease to other individuals.

In an exemplary embodiment, the disclosed antibody/IgY administration is performed to inoculate against or otherwise prevent the SARS-CoV-2 virus. For instance, IgY that is collected from immunized hens as part of the disclosed process, can be used as a therapeutic for individuals who are symptomatic, in conjunction with antivirals or systemic passive immunity to aid in clearance of the virus from the nasal cavity/GI tract and reducing transmission. IgY may be useful in subjects who test positive for the virus but are asymptomatic/pre-symptomatic and; may decrease the number of such patients progressing to symptomatic, progressive infection.

The disclosed IgY remedy may reduce the risk of such subjects transmitting the virus. Administering IgY from collected eggs could be used in combination with systemic antivirals and/or convalescent serum to help eradicate nasopharyngeal virus in sick hospitalized patients. Nasal IgY can be used as prophylaxis against nasal colonization during upticks or future outbreaks of SARS-CoV-2, even in subjects who have been vaccinated, given that systemic vaccination does not prevent subsequent nasal colonisation. It can be a bridging solution for all, pending a global vaccine. The disclosed nasal IgY administration may be particularly useful for prophylaxis in high risk subjects, such as people with inadequate responses to a vaccine, nursing home residents, international travellers, sailors or passengers on ships at sea or others working or living in crowded, confined spaces, and critical workers with repeated exposures.

In various embodiments, the IgY product may be complementary to other passive immune approaches such as convalescent serum or recombinant polyclonal human antibodies. For example, it can be used to help clear virus from the nasopharyngeal surface of patients with systemic infection (i.e. lung or other organs involved) who are at the same time receiving convalescent serum to help clear the systemic infection

At step 105 of the process 100 for immunizing egg laying chickens to produce IgY containing eggs, female chicks are immunized with one or more antigens. Administration of antigens disclosed herein may be performed by various means including but not limited to spraying chickens with an antigen composition, including antigen in chicken feed, including antigens in water that is provided to chickens. As shown in FIG. 1, antigen may also be administered by syringe. However, a traditional farm setting is more likely to administer antigen via a method other than injection. The antigens cause the chicks to produce antibodies that target the antigen. At step 110 of the process, the hens are immunized one or more times with the same antigen as step 105. The second immunization causes the hen to produce more antibodies and/or produce the antibodies over a longer period of time.

At step 115, the hen may be fed a diet that has been shown to increase production of IgY antibodies beyond what the hen would produce from immunization alone. In an exemplary embodiment, the hen is fed a diet that includes supplementation of selenium, vitamin D, vitamin A, and vitamin E in any of the amounts or concentrations disclosed herein. The high-nutrient diet increases production of IgY antibodies in the hen, which results in increased IgY in eggs that are later collected from the hen.

At step 120, eggs are collected from the hen. According to standard agricultural practice, the hen may lay 1 egg per day. In various embodiments, IgY eggs may be collected about 6-8 weeks after the first immunization. Accordingly, IgY antibodies may be produced within about 2 months from detecting a new pathogen through the process 100 of immunizing hens and collecting IgY from eggs. The high nutrient diet may be continually fed to the hen as it produces eggs according to the process 100.

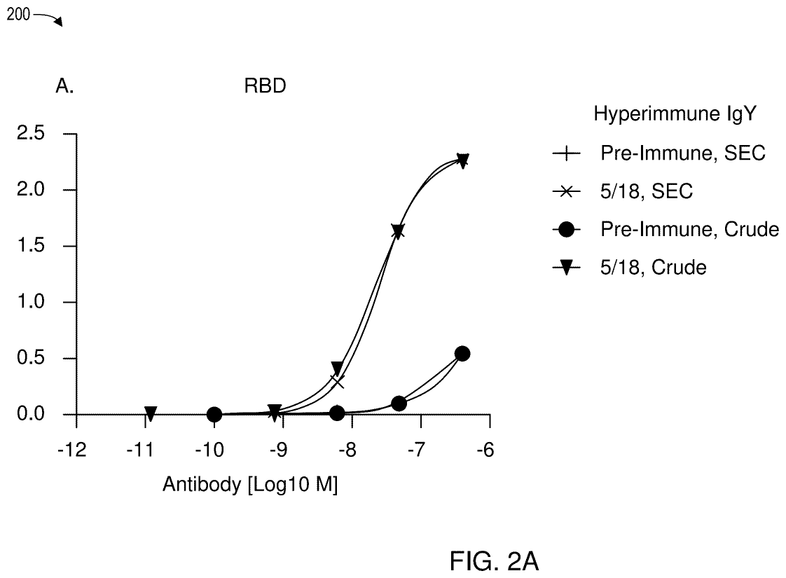

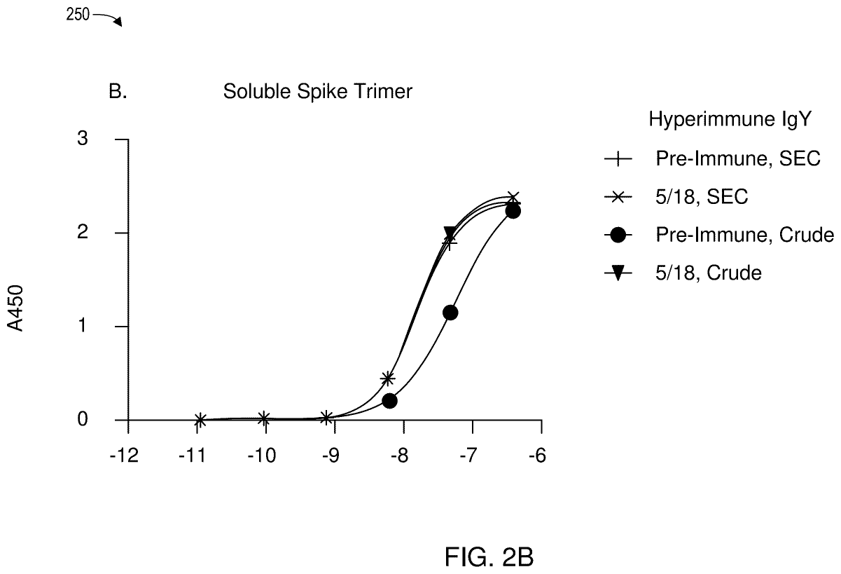

Referring to FIG. 2A, FIG. 2A is an ELISA assay 200 showing reactivity of the receptor binding domain (RBD) for SARS-CoV-2 and IgY antibodies derived from various sources including unimmunized purified eggs via size exclusion chromatography (SEC), immunized purified eggs via SEC, unimmunized crude eggs, and immunized crude eggs.

The graph 200 shows that all IgY antibodies collected from eggs showed at least minimal affinity for RBD SARS-CoV-2. IgY antibodies were collected from eggs of hens that were not immunized and hens that were immunized to Avian Infectious Bronchitis Virus (IBV). Further, each of the immunized and non-immunized groups were divided into a crude sample and a purified sample.

The two non-immune samples, labeled “Pre-Immune Crude” and “Pre-Immune, SEC, showed similar affinity to RBD SARS-CoV-2. The two samples from the eggs of immunized hens, samples labeled “ 5/18, Crude” and “ 5/18, SEC” showed higher affinity then the non-immunized samples.

The disclosed subject matter is a IgY-boosted shell egg from chickens that is specially formulated to strengthen the immune system of an individual. The IgY-boost hens are fed a high-nutrient dietary supplement and undergo a boosted vaccination regime which results in the eggs that contain increased levels of immuno-proteins which strengthen the immune system and decrease the chance of viral infection and transmission of viruses.

The drawing shows a scientific diagram titled “6-8 Week Response For Antibodies Against New Pathogen”. The diagram shows a timeline of the response of antibodies against a new pathogen, starting at T=0 and ending at 6-8 weeks. The diagram shows a group of chickens at T=0 and at 6-8 weeks, with the chickens at T=0 shown without any antibodies and the chickens at 6-8 weeks shown with antibodies (IgY) against the new pathogen.

The IgY immunoglobulin antibodies are present in the yolk of these specially produced IgY-boost eggs. Oral ingestion of those same immunoglobins and micronutrients in eggs can provide significant health benefits. Chicken antibodies, known as IgY immunoglobulins are similar in structure to IgG antibodies and compatible for human use, but more stable than IgG antibodies and exhibit survival during passage through the GI track.

Egg-derived antibodies are polyclonal and differ from monoclonal antibodies in that they contain multiple antibody species which can cross react with multiple epitopes from related species; thus, veterinary immunogens can provide protection against human pathogens. Ingestion of the eggs containing IgY antibodies have been shown to be effective in prevention and treatment of disease in various locations within the gastrointestinal tract including tooth decay, periodontitis and gingivitis, gastritis and gastric ulcer, celiac disease, oral thrush, and infant rotavirus diarrhea.

Recent results from a controlled study indicate that the IgY-boost eggs contain an average of 20% more IgY antibody than commercial eggs. When hens are immunized against a pathogen, the IgY antibodies are produced in the blood serum and passed to the embryo through the egg yolk. The IgY-eggs are collected from hens fed specially formulated high nutrient diets containing selenium, vitamins A, & D.

Referring to FIG. 2B, FIG. 2B is an ELISA assay 250 showing reactivity of the soluble spike trimer for SARS-CoV-2 and IgY antibodies derived from various sources including unimmunized purified eggs via size exclusion chromatography (SEC), immunized purified eggs via SEC, unimmunized crude eggs, and immunized crude eggs. The IgY sample labeled “Pre-Immune, Crude”, which is the non-immunized and non-purified sample, had the lowest affinity for the spike trimer of SARS-CoV-2. The other three IgY samples labeled “Pre-Immune, SEC” (non-immunized, purified), “ 5/18, SEC” (immunized, purified), and “ 5/18, Crude” (immunized, non-purified) all showed similar affinity for the spike trimer of SARS-CoV-2.

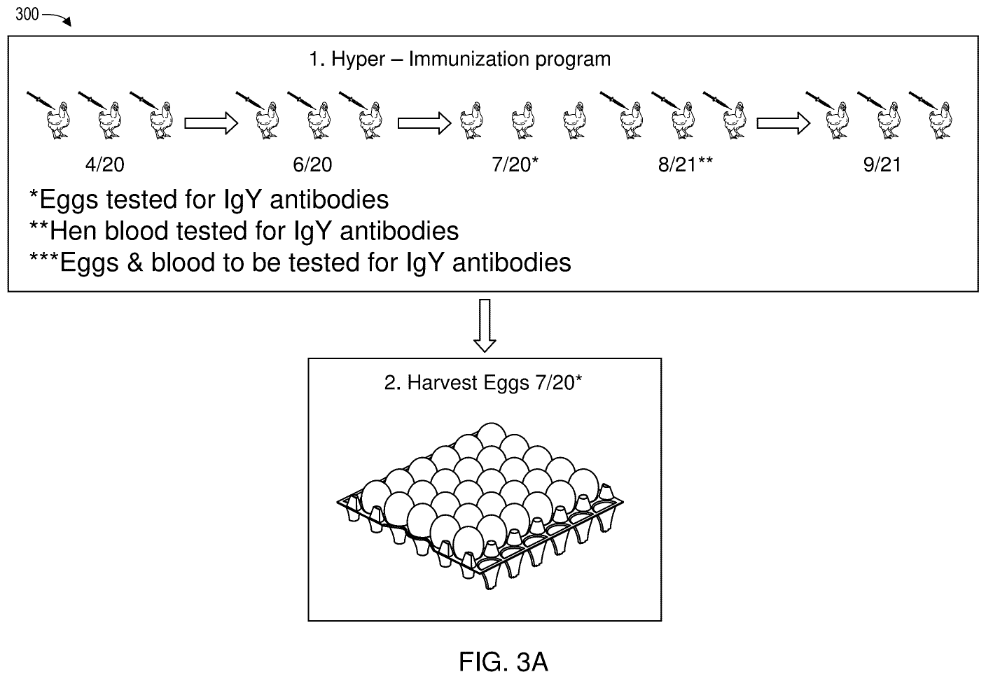

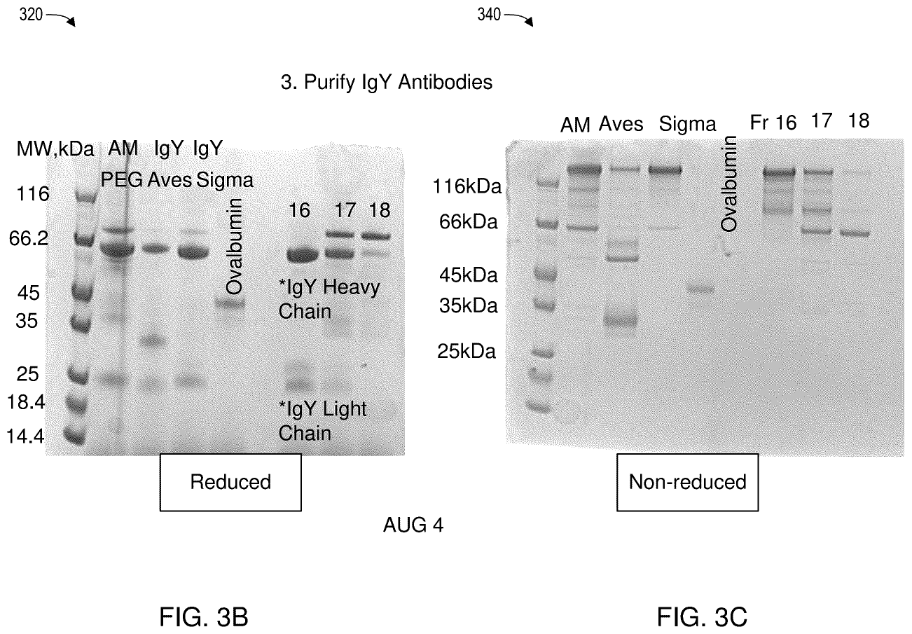

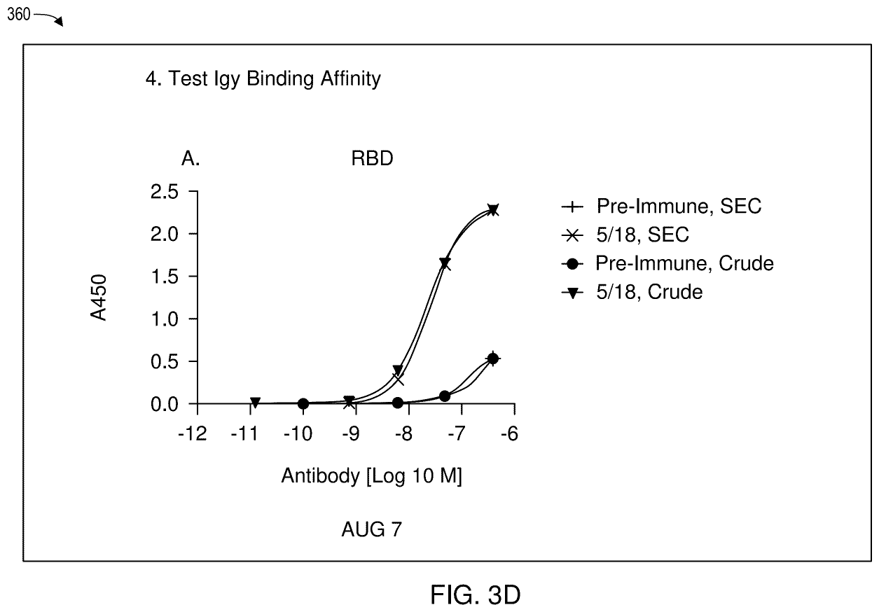

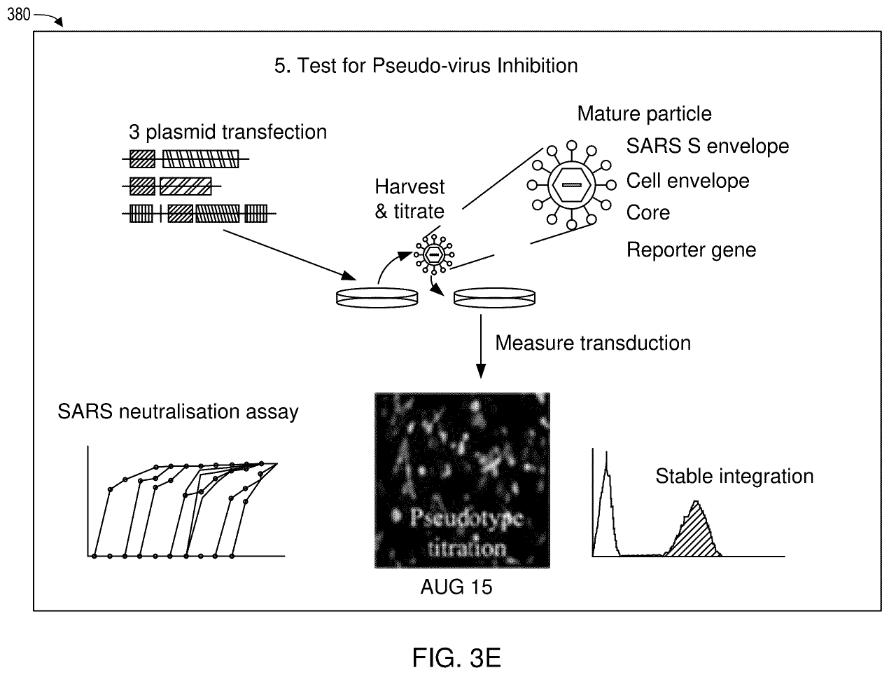

Referring to FIG. 3A, FIG. 3A is a schematic 300 showing an embodiment of a system for producing IgY boosted eggs via a hyperimmunization program. Hens may be immunized (or boosted) multiple times to increase the amount and concentration of antibodies in their blood and in eggs that they produce. In various embodiments, the hens are immunized to the same antigen on a regular schedule. In an exemplary embodiment, the hens are immunized to Avian Infectious Bronchitis Virus (IBV), which is related to SARS-CoV-2.

As shown in the schematic 300, hens are immunized at 2-month intervals. They are further regularly tested for IgY antibodies. And further yet, eggs that are laid by the hens are regularly tested for IgY antibodies. In various embodiments, the frequency of the immunization has a shorter interval between immunizations. In various embodiments, the frequency of the immunization has a longer than 2-month interval between immunizations.

In various embodiments, the frequency of immunization is dependent on antibody testing of the hens, For instance, a frequency of immunization may be increased in hens with relatively low amounts of antibodies in their blood tests. Likewise, a frequency of immunization may be decreased in hens with relatively high amounts of antibodies in their blood tests.

In an exemplary embodiment, the frequency of immunization is dependent on antibody testing of the eggs. For example, a frequency of immunization may be increased in bens who's eggs are tested to have relatively low amounts of antibodies. Similarly, a frequency of immunization may be decreased in hens who's eggs are tested to have relatively high amounts of antibodies. In an exemplary embodiment, a frequency of immunization may be dependent on both a blood test for antibodies and a test for antibodies collected from eggs laid by the hen.

For example, hens may be inoculated any time their blood sample shows an IgY concentration of below about 10 mg/mL. In various embodiments, hens are inoculated when their blood sample shows an IgY concentration of below about 11 mg/mL. In various embodiments, hens are inoculated when their blood sample shows an IgY concentration of below about 12 mg/mL. In various embodiments, hens are inoculated when their blood sample shows an IgY concentration of below about 13 mg/ml. In various embodiments, hens are inoculated when their blood sample shows an IgY concentration of below about 14 mg/mL. In various embodiments, hens are inoculated when their blood sample shows an IgY concentration of below about 15 mg/mL. In various embodiments, hens are inoculated when their blood sample shows an IgY concentration of below about 16 mg/mL.

In various embodiments, the IgY in blood concentration is determined between about once every day and about once every 4 weeks. In various embodiments, the IgY in blood concentration is determined between about once every day and about once every 2 months. In various embodiments, the IgY in blood concentration is determined between about once every 3 days and about once every 4 weeks, In various embodiments, the IgY in blood concentration is determined between about once every week and about once every 4 weeks. In various embodiments, the IgY in blood concentration is determined between about once every two weeks and about once every 4 weeks. In various embodiments, the IgY in blood concentration is determined between about once every week and about once every 4 weeks, In various embodiments, the IgY in blood concentration is determined between about once every two weeks and about once every 6 weeks. In various embodiments, the IgY in blood concentration is determined between about once every two weeks and about once every 10 weeks.

Hens can be immunized multiple times to increase the amount and concentration of antibodies in their blood and the eggs they produce. In various embodiments, hens are immunized with the same antigen on a regular schedule, For example, hens may be immunized with Avian Infectious Bronchitis Virus (IBV), which is related to SARS-CoV-2.

As shown in schematic 300, hens are immunized at intervals of 2 months. They are also regularly tested for IgY antibodies, and eggs laid by the hens are also regularly tested for IgY antibodies. In some embodiments, the frequency of immunization may have a shorter interval between immunizations, while in others it may have an interval longer than 2 months.

In some embodiments, the frequency of immunization depends on antibody testing of the hens. For example, the frequency of immunization may be increased in hens with relatively low amounts of antibodies in their blood tests, while it may be decreased in hens with relatively high amounts of antibodies in their blood tests, In an exemplary embodiment, the frequency of immunization depends on antibody testing of the eggs. For instance, the frequency of immunization may be increased in hens whose eggs are tested to have relatively low amounts of antibodies, while it may be decreased in hens whose eggs are tested to have relatively high amounts of antibodies. In another exemplary embodiment, the frequency of immunization may depend on both a blood test for antibodies and a test for antibodies collected from eggs laid by the hen.

For instance, hens may be inoculated any time their blood sample shows an IgY concentration below about 10 mg/mL. In various embodiments, hens are inoculated when their blood sample shows an IgY concentration below about 11 mg/mL or below about 12 mg/mL or below about 13 mg/mL or below about 14 mg/mL or below about 15 mg/mL or below about 16 mg/mL. In various embodiments, the IgY concentration in blood is determined between about once every day and about once every 4 weeks or between about once every day and about once every 2 months or between about once every 3 days and about once every 4 weeks or between about once every week and about once every 4 weeks or between about once every two weeks and about once every 4 weeks or between about once every two weeks and about once every 6 weeks or between about once every two weeks and about once every 10 weeks.

Referring to FIG. 3B and FIG. 3C, FIG. 3B is an image of an SDS-PAGE analysis 320 in reducing agent comparing various commercial IgY samples with purified IgY fractions collected from hens that were immunized according to the disclosed subject matter. FIG. 3C is an image of an SDS-PAGE analysis 340 in non-reducing agent comparing various commercial IgY samples with purified IgY fractions collected from hens that were immunized according to the disclosed subject matter. The SDS-PAGE analyses in FIGS. 3B and 3C have controls of IgY samples from the commercial sources Active Motif, Aves Labs, and Sigma. An ovalbumin sample is included in both figures.

Referring to the SDS-PAGE analysis 320 in FIG. 3B, the Active Motif sample shows a band just above 66.2 kDa, a band at 66.2 kDa, a barely visible band at 45 kDa, a band at 35 kDa, and a band at 25 kDa. The Aves Labs IgY sample shows a barely visible band just above 66.2 kDa, a band at 66.2 kDa, a band between 25 and 35 kDa, and a band at 25 kDa. The Sigma IgY sample shows a band just above 66.2 kDa, a band at 66.2 kDa, and a band at 25 kDa. The ovalbumin sample shows a band at 45 kDa and a barely visible band between 35 kDa and 45 kDa.

The SDS-PAGE gel for IgY fraction 16 shows a band at 66.2 kDa, a band just above 25 kDa, and a band at 25 kDa. IgY fraction 17 shows a band just above 66.2 kDa, a band at 66.2 kDa, a barely visible band around 45 kDa, two barely visible bands around 35 kDa, a band just above 25 kDa, and a band at 25 kDa. IgY fraction 18 shows at band just above 66.2 kDa, a band at 66.2 kDa, a barely visible band around 45 kDa, and two barely visible bands around 35 kDa. There is little overlap between fractions 16 and 18 where fraction 16 has lighter weight proteins and fraction 18 has just one strong band just above 66.2 kDa.

Referring to the SDS-PAGE analysis 340 in FIG. 3C, the Active Motif sample shows a band above 116kDa, a band between 66 kDa and 116 kDa, three barely visible bands between 45 kDa and 66 kDa, a barely visible band at 35 kDa, and a barely visible band just below 35 kDa.

The Aves Labs IgY sample shows a band above 116 kDa, a barely visible band at 66 kDa, a barely visible band between 66 kDa and 116 kDa, a barely visible band at 66 kDa, two bands between 45 kDa and 66 kDa, a band just below 35 kDa and a band between 25 kDa and 35 kDa. The Sigma IgY sample shows a band above 116 kDa, a barely visible band at 116 kDa, two barely visible bands between 66 kDa and 116 kDa, and a band at 66 kDa. The ovalbumin sample shows a band between 35 kDa and 45 kDa and a band at 35 kDa.

The SDS-PAGE gel for IgY fraction 16 shows a band above 116 kDa, a band at 116kDa, a band between 66 kDa and 116 kDa, and a band at 66 kDa. IgY fraction 17 shows a band above 116 kDa, a band at 116 kDa, a band between 66 kDa and 116 kDa, a band at 66 kDa, two barely visible bands between 45 kDa and 66 kDa, and a barely visible band at 35 kDa. IgY fraction 18 shows at band above 116 kDa, a band between 66 kDa and 116 kDa, a band at 66 kDa, a barely visible band between 45 kDa and 66 kDa, and two barely visible bands around 35kDa. There is little overlap between fraction 16 and fraction 18 where fraction 16 contains more heavy molecular weight proteins and fraction 18 has lighter molecular weight proteins.

Referring to FIG. 3D, FIG. 3D is an ELISA assay 360 showing reactivity of the receptor binding domain (RBD) for SARS-CoV-2 and IgY antibodies derived from various sources including unimmunized purified eggs via size exclusion chromatography (SEC), immunized purified eggs via SEC, unimmunized crude eggs, and immunized crude eggs. Both of the unimmunized samples, “Pre-Immune, SEC” and “Pre-Immune, Crude”, had similar affinity for RBD SARS-CoV-2. The immunized samples, “ 5/18, SEC” and “ 5/18, Crude”, had higher affinity than the unimmunized samples. There was no noticeable difference between affinity of the immunized samples.

Referring to FIG. 3E, FIG. 3E is an embodiment of a process 380 for creating a pseudo-virus neutralization assay, with which to test effectiveness of derived IgY from immunized hens, for neutralizing SARS-CoV-2. The process 380 includes transfecting a host cell with the well established 3 plasmid technique. The host cell then produces a pseudo-virus, which in this case based on SARS-CoV-2. The SARS-CoV-2 pseudo-virus is harvested. In various embodiments, the neutralization assay comprises cells that are infected with a mixture of the SARS-CoV-2 pseudo-virus and a serum of IgY taken from eggs of hens that were immunized. Results of the neutralization assay may determine the IgY is capable of neutralizing SARS-CoV-2.

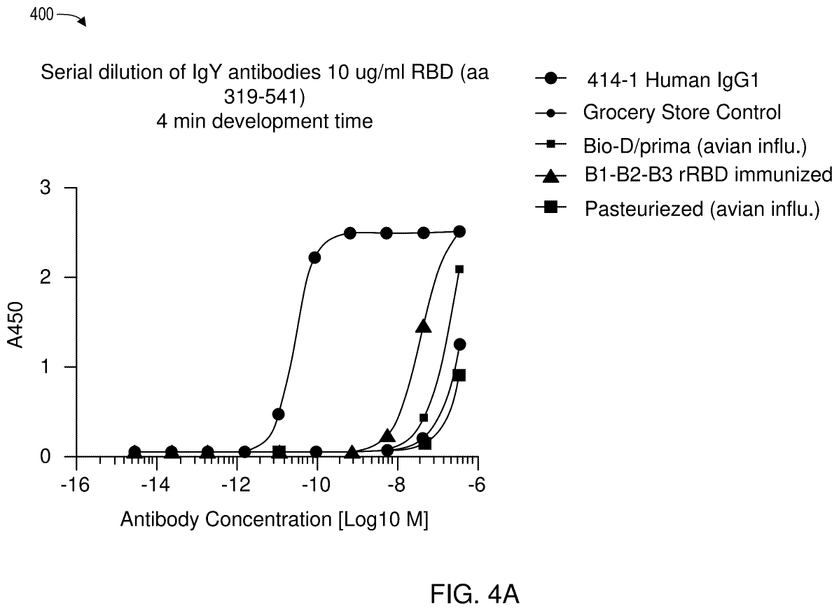

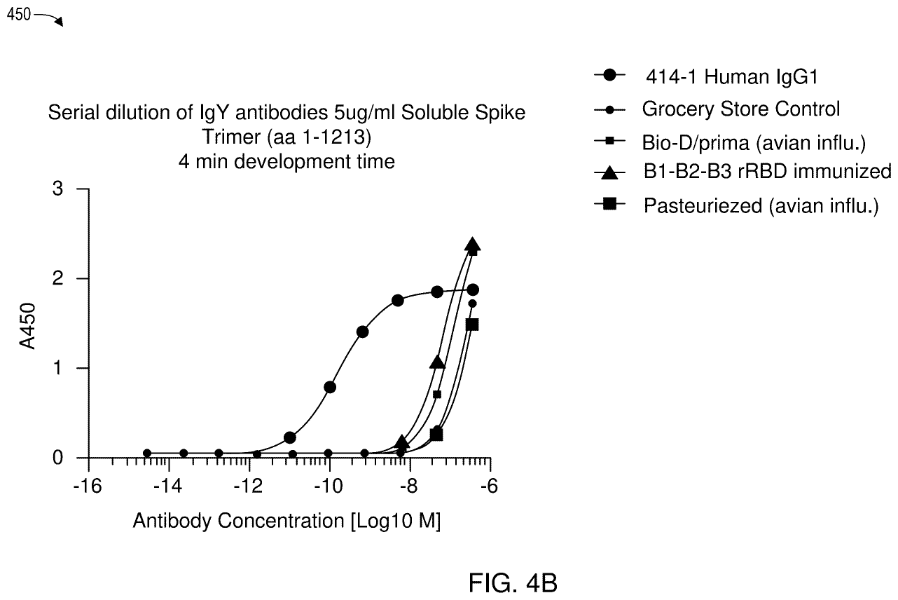

Referring to FIG. 4A, FIG. 4A is an ELISA assay 400 showing reactivity of the receptor binding domain (RBD) for SARS-CoV-2 and immunoglobulin samples representing human IgG1 (414-1 Human IgG1), IgY derived from grocery store eggs (control), eggs from hens that were supplemented with vitamin D and immunized to avian influenza, eggs from hens that were supplemented with vitamins B1, B2, and B3 and immunized to the recombinant receptor binding domain (rRBD) of SARS-CoV-2 (B1-B2-B3 rRBD immunized), and pasteurized eggs that were immunized to avian influenza.

Among the samples shown in the ELISA assay 400, the 414-1 Human IgG1 sample had the highest affinity to SARS-CoV-2 RBD (aa 319-541). The B1-B2-B3 rRBD immunized sample showed the next highest affinity to SARS-CoV-2 RBD. The sample supplemented with vitamin D had the next highest affinity. The sample taken from a grocery store egg had the next highest affinity. The pasteurized egg that was immunized to avian influenza had the lowest affinity to SARS-CoV-2 RBD.

Referring to FIG. 4B, FIG. 4B is an ELISA assay showing reactivity of the soluble spike trimer for SARS-CoV-2 and immunoglobulin samples representing human IgG1, IgY derived from grocery store eggs (control), eggs from hens that were supplemented with vitamin D and immunized to avian influenza, eggs from hens that were supplemented with vitamins B1, B2,and B3 and immunized to the recombinant receptor binding domain (rRBD) of SARS-CoV-2 (B1-B2-B3 rRBD immunized), and pasteurized eggs that were immunized to avian influenza.

Among the samples shown in the ELISA assay 450, the 414-1 Human IgG1 sample had the highest affinity to the spike trimer for SARS-CoV-2 (aa 1-1213). The B1-B2-B3 rRBD immunized sample showed the next highest affinity to the spike trimer for SARS-CoV-2. The sample supplemented with vitamin D had the next highest affinity, The samples from the grocery store egg and the sample from the pasteurized egg laid by a hen immunized to avian influenza both had the lowest affinity to the spike trimer for SARS-CoV-2.

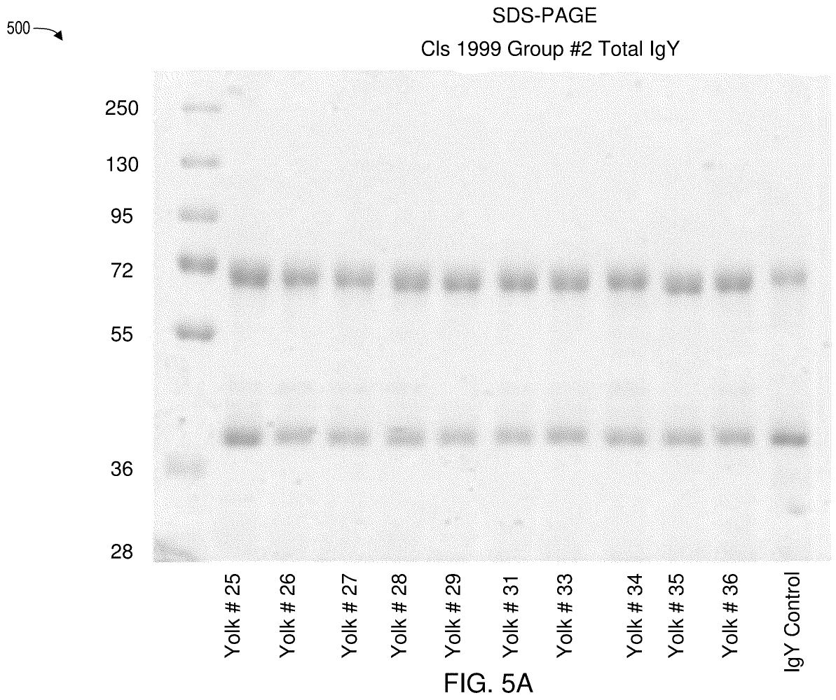

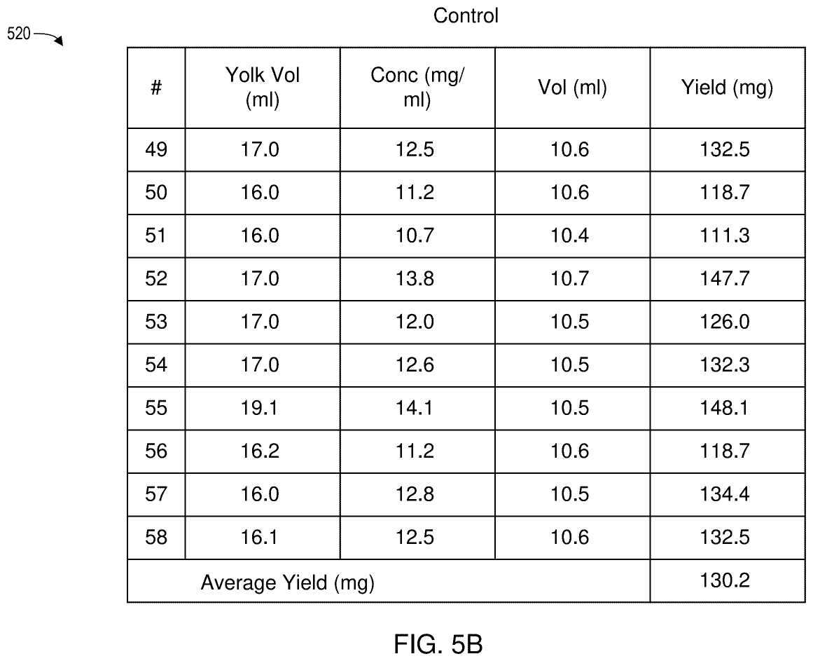

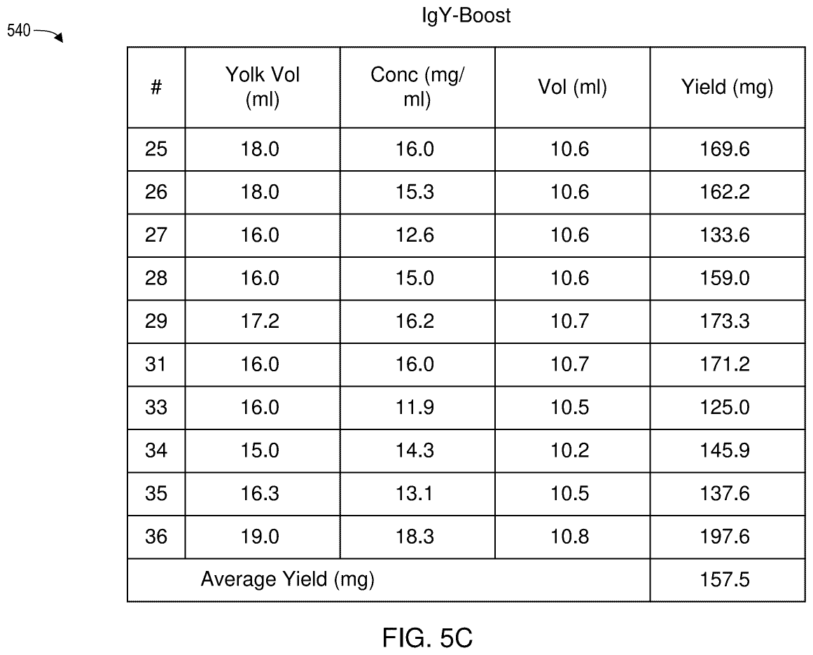

Referring to FIG. 5A, FIG. 5A is an SDS-PAGE analysis 500 of immunoglobulin samples taken from the yolk of eggs laid by immunized hens. Each of the yolk samples in the gel including Yolk #25, Yolk #26, Yolk #27, Yolk #28, Yolk #29, Yolk #31, Yolk #33, Yolk #34, Yolk #35, and Yolk #36 had bands at 72 kDa and bands just above 36 dDa. Similarly, the IgY control had a band at 72 kDa and a band just above 36 dDa. Accordingly, all IgY samples in the SDS-PAGE analysis 500 had similar protein composition.

Referring to FIGS. 5B and 5C, FIG. 5B is a table 520 showing a yield of IgY from eggs laid by hens from a control group. FIG. 5C is a table 540 showing a yield of IgY from eggs laid by hens that were immunized and fed a diet based on an embodiment of the disclosed subject matter. Hens were fed a high-nutrition diet and immunized multiple times to increase a total amount of IgY in eggs that were laid by the hens. The increased IgY effectively lowers the cost and decreases the time to produce IgY from chickens.

The data from the table 520 in FIG. 5B and the table 540 in FIG. 5C show that hens that were immunized and fed a diet according to the disclosed subject matter produced approximately 21% more IgY than a control group. Refinement of the process by adjusting nutrient concentrations of selenium, vitamin D, vitamin A, and vitamin E, and adjusting a number of immunizations and frequency of immunizations may further increase the yield of IgY extracted from eggs produced by hens according to the disclosed process.

Many variations may be made to the embodiments described herein. All variations, including combinations of embodiments, are intended to be included within the scope of this disclosure. The description of the embodiments herein can be practiced in many ways. Any terminology used herein should not be construed as restricting the features or aspects of the disclosed subject matter. The scope should instead be construed in accordance with the appended claims.

1. A method for increasing IgY in a bird egg, the method comprising:
exposing a bird to one or more antigens, wherein the exposing is performed on at least two separate occurrences; and
wherein a time between the at least two separate occurrences is at least one week.

2. The method of claim 1, further comprising determining an amount of IgY in blood of the bird prior to exposing the one or more antigens on at least one occasion; and
wherein a time of the exposing the one or more antigens on at least one occasion is based on the amount of IgY in the blood.

3. The method of claim 2 wherein the determining an amount of IgY is performed at least twice.

4. The method of claim 3, wherein the determining an amount of IgY is performed at a frequency of between about once every day and once every 4 weeks.

5. The method of claim 2, wherein a frequency between the at least two separate occurrences of the exposing the one or more antigens is between about one time a month and about one time every two months.

6. The method of claim 1, further comprising administering the bird egg to a human patient or animal subject.

7. The method of claim 6, wherein the administering comprises oral administration of the bird egg.

8. The method of claim 2, wherein the bird egg has about 20% more IgY antibodies than control eggs.

9. The method of claim 8, wherein the IgY antibodies are used to inoculate against a disease within a gastrointestinal tract in a human patient or animal subject.

10. The method of claim 9, wherein the disease within the gastrointestinal tract is selected from the group consisting of tooth decay, periodontitis and gingivitis, gastritis and gastric ulcer, celiac disease, oral thrush, and infant rotavirus diarrhea.

11. An IgY delivery system, the system comprising:
an egg that is laid by a bird that was exposed to one or more contagions;
wherein the bird was exposed to the one or more contagions on two or more occasions.

12. The system of claim 11, wherein at least one of the two or more occasions occurs more than about 16 weeks after the bird hatched.

13. The system of claim 11, wherein an amount of IgY in blood of the bird was tested prior to at least one of the two of more occasions.

14. The system of claim 13, wherein a time of at least one of the two or more occasions is based on the amount of IgY in the blood of the bird.

15. The system of claim 14, wherein the time of at least one of the two or more occasions is triggered when an IgY concentration in the blood dropped below about 13 mg/mL.

16. The system of claim 13, wherein the egg is prepared by purifying IgY from yolk of the egg; and
wherein the IgY is bound to at least one of S1 and S2 subunits from a Delta variant of the COVID virus.

17. The system of claim 16, wherein the one or more contagions comprise avian infectious bronchitis virus (IBV).

18. The system of claim 13, wherein the bird is verified to contain IgY in its blood prior to the bird laying the egg.

19. A method of producing IgY, the method comprising:
administering a contagion to a bird, the contagion comprising avian infectious bronchitis virus (IBV), two or more times;
collecting eggs from the bird; and
extracting IgY from the eggs.

20. The method of claim 19, wherein the administering comprises determining a concentration of IgY in blood of the bird prior to at least one of the two or more times; and
and determining that the concentration of IgY in the blood dropped below about 13 mg/mL.
